(19) United States
(12) Patent Application Publication
(10) Pub. No.: US 2005/0261474 A1
(43) Pub. Date: Nov. 24, 2005

(54) Method of support-based chemical synthesis

(75) Inventors: Richard A. Houghten, Solana, CA (US); Yongping Yu, San Diego, CA (US)

Correspondence Address: WELSH & KATZ, LTD, 120 S RIVERSIDE PLAZA, 22ND FLOOR, CHICAGO, IL, 60606, US

(73) Assignee: Mixture Sciences, Inc.

(21) Appl. No.: 10/850,336

(22) Filed: May 20, 2004

(51) Int. Cl. C07H 21/04; C07K 1/02; C08B 37/00

(52) U.S. Cl. 530/333; 536/25.3; 536/123

(57) ABSTRACT

A method of synthesis on a solid phase support is disclosed that provides a cleaved product containing a protecting group that would have been cleaved by reaction with anhydrous HF wherein the support is volatilized during cleavage of the protected product from the support by reaction with diluted HF.

### TECHNICAL FIELD

The invention relates to a method of chemical synthesis that takes place on a support that can be a solid or a liquid. More particularly, the invention pertains to support-based synthetic methods utilizing chemical reagents that volatilize the support during cleavage of the product from the support. The cleavage and volatile-formation reactions are carried out under conditions that permit the retention of one or more protecting groups on the synthesized product after the cleavage reaction where those one or more protecting groups would have been cleaved had anhydrous HF been used for the cleavage reaction.

### BACKGROUND OF THE INVENTION

The preparation of compounds using a solid phase approach was first described by Merrifield in 1963 [Merrifield, 1963, J. Am. Chem. Soc., 85:2149-2154.] Since this initial seminal concept in which a polystyrene support was used to prepare peptides, a wide range of different supports have been used (i.e., polyamides [Atherton et al., 1975, J. Am. Chem. Soc., 97:6584-6585], porous glass [Parr et al., 1974, Justus Liebigs Ann. Chem., pp. 655-666] and microchip quartz [Fodor et al., 1991, Science, 251:767-773]). Although useful, these supports all require a final cleavage step in which the compounds (peptides, peptidomimetics, oligonucleotides, small organic molecules, various heterocycles, and the like) are cleaved from the support, then separated from the spent support.

Where the compound of interest can be used in an immobilized manner (i.e., it remains on the support in its final use and/or manifestation), then the remaining support may not be problematic, and in fact may be useful for certain assays. However, in the majority of cases, the compound of interest is used in solution and therefore has to be separated from its support. Significant time, increased yield, and/or cost savings can be realized if the removal of the support did not have to be accomplished in a separate step following cleavage of the desired compound from the support (typically by filtration or centrifugation).

In addition, it is often desirable to prepare by solid phase chemistries materials having their protecting groups intact, as in N- or C-terminally protected or side chain-protected peptide fragments or other compound types (e.g.; benzyl ester hydantoins) an in which one desires to incorporate “protecting” groups as an integral component(s) of the desired final product that can be used in the synthesis of larger peptides, proteins, or peptidomimetics.

Although the preparation of compounds using a solid phase approach with volatilization of the solid support has been described in U.S. Pat. No. 6,476,191, the products of the synthetic method that uses pure HF disclosed in that patent are without their protecting groups, all such groups typically being lost during the cleavage-support volatilization step.

The invention disclosed hereinafter provides one solution to the problem of separating the spent support from the desired synthesized material while maintaining at least some of the protecting groups on the product.

### BRIEF SUMMARY OF THE INVENTION

The present invention contemplates synthesis of a protecting group-containing product on a siliceous support where the support is volatilized upon completion of synthesis by reaction with diluted hydrofluoric acid (HF) as defined hereinafter, and under conditions in which at least one of the protecting groups of the product that would have been cleaved by anhydrous HF remains bonded to the synthesized product after cleavage of the product from the support.

Thus, a siliceous support-based (solid or liquid phase support) synthesis method is contemplated in which at least one reagent containing a protecting group is coupled to a siliceous support. A plurality of reactions is carried out upon the protected reagent coupled to the support to form a protected product coupled to the support that is then cleaved to form the soluble or insoluble product by reaction with HF. The improvement in this synthesis is that during cleavage, the siliceous support is reacted with diluted HF to form a volatile compound(s) that is separated from the desired product by vaporization as by distillation. The reaction with diluted HF is carried out under conditions such that at least one protecting group that would have been cleaved by reaction with anhydrous HF remains bonded to the cleaved product. That at least one protecting group that would remain bonded to the product need not be identical to the protecting group present prior to cleavage, but nevertheless, still acts as a protecting group and is selectively removable.

Additionally, the present invention contemplates a method for solid phase synthesis of a product that comprises coupling a first reagent to a siliceous support to form a support-coupled first reagent. The support-coupled first reagent is reacted with a second reagent, which can be the same or a different reagent and wherein one or both of the first and second reagents contain at least one protecting group to form a protected support-coupled product. The protected support-coupled product is cleaved from the support to form a cleaved product by reaction with diluted HF. The reaction with diluted HF is carried out under conditions such that at least one protecting group that would have been cleaved by reaction with anhydrous HF remains bonded to the cleaved product.

A particularly preferred siliceous support is silica itself. Cleavage of the product from the support and formation of the volatile compound is typically carried out in a single step, although separate steps can be used.

A particularly preferred diluted HF reagent for the cleavage of the product from the support while retaining at least one of the product's protecting groups that would have been cleaved were anhydrous HF used is a mixture of about 5 percent to about 50 percent hydrogen fluoride in water. Another preferred diluted HF is 10 to about 70 percent HF in 90 to about 30 percent pyridine. A third preferred diluted HF is about 5 to about 50 percent HF in about 95 to about 50 percent dimethylsulfide.

The present invention has several benefits and advantages.

One benefit is the simplicity in reaction steps because the usual filtering or centrifugation step is not required thereby saving time, effort, and money.

Another advantage is that losses of the desired product that can occur because of entrapment of the desired product within the usual spent support, or within the manipulation of filtration and centrifugation do not occur.

An additional benefit is that products can be synthesized and cleaved from the support while still having their protecting groups intact.

Still further benefits and advantages of the contemplated invention will be apparent to the skilled worker from the disclosure that follows.

### BRIEF DESCRIPTION OF THE DRAWINGS

In the drawings forming a part of this invention,

FIG. 1 in two panels as FIG. 1A and FIG. 1B is the HPLC (1A)/MS (1B) print-out of the crude material from the synthesis of the C-terminal benzyl ester of L-valine-L-alanine-L-phenylalanine was that was prepared on phenylmethylchloro silica gel and using standard Boc peptide synthesis chemistry with removal of the N-terminal Boc group with TFA, and volatilization of 100 mg of silica by treatment with 10% HF in water (4.0 ml) at room temperature for one hour (shown as M+Na).

FIG. 2 shows a structural formula of Vancomycin, an oligosaccharide.

FIG. 3 in two panels as FIG. 3A and FIG. 3B, shows the HPLC results for Vancomycin itself (3A) and Vancomycin (3B) after treatment with 10 percent HF in water at room temperature overnight (about 18 hours). The small peak seen at 2.15 minute is vancomycin minus it's glycol unit, thus indicating that less than 5% of the glycol unit was lost in 18 hours (it should be noted that volatilization is frequently accomplished in less than 1-2 hours at room temperature).

FIG. 4 in two panels as FIG. 4A and FIG. 4B on the left side (FIG. 4A) shows a post solid support cleavage and vaporization HPLC elution pattern for the O-benzyl hydantoin product shown on the right side (FIG. 4B) in the mass spectrum (shown as M+Na).

FIG. 5A and FIG. 5B are the HPLC (FIG. 5A)/MS (FIG. 5B) of the crude material resulting from the synthesis of the C-terminal N-benzyl amine of L-tyrosine(BrZ)-L-tyrosine(BrZ)-L-phenylalanine-L-proline prepared on p-benzylamine silica gel and using standard Boc peptide synthesis chemistry (utilizing removal of the N-terminal Boc group with TFA and volatilization of silica by treatment with 10% HF in water at room temperature for one hour).

FIG. 6A and FIG. 6B show the RP-HPLC and MS results for a post solid support cleavage and volatilization of the resulting polyamine following diborane reduction.

### DETAILED DESCRIPTION OF THE INVENTION

A synthetic method is contemplated in which usual support-based synthetic steps are carried out in the synthesis of a protected product (i.e., a product bonded to at least one protecting group) such as a peptide, peptidomimetic amine, glycopeptide, oligonucleotide, oligosaccharide or heterocyclic product as noted hereinafter using a Merrifield synthesis or the like. Illustrative, traditional, solid phase syntheses of such materials can be seen in U.S. Pat. No. 4,631,211, No. 5,369,017, No. 5,504,190, No. 5,480,971, No. 5,846,731, No. 6,197,529, No. 5,556,762, No. 6,441,172, and No. 6,545,032.

Several model products have been examined using a cleavage/volatile siliceous support-forming reaction using diluted HF. Thus, peptides and several small molecule heterocycles have shown substantially complete stability under a variety of conditions. Vancomycin, an oligosaccharide-containing drug was found to be more than 95 percent stable to contact with 10 percent HF in water after 24 hours, whereas a majority of the disaccharide was cleaved after 18 hours using 10 percent HF in dichloromethane. A glycopeptide containing a serine-linked GalNAc group was completely stable when treated with 10 percent HF water for 18 hours at room temperature. About 20 percent of poly-adenylic acid was lost after a 2-hour treatment at room temperature in 10 percent HF in water.

The improvements here lie in the cleavage of the protected product from the siliceous solid or liquid support with diluted hydrofluoric acid (HF), and the separation of the cleaved protected product from the support by conversion of the siliceous support into a volatile material by reaction with diluted HF, with the volatile material being separated from the desired reaction product by vaporization, e.g., at atmospheric pressure or below. Thus, the usually used filtration or extraction of the desired product from the spent support is unnecessary. The reaction with diluted HF is carried out under conditions such that at least one protecting group that would have been cleaved by reaction with anhydrous HF remains bonded to the cleaved product.

Taking solid phase peptide synthesis as exemplary, at least one reagent such as a side chain- and N-protected amino acid is coupled to the support. A plurality of reactions is carried out on that support-coupled reagent such as N-deprotection, coupling of another side chain- and N-protected amino acid to form a support-coupled reaction product. The linkage between the support and desired peptide product is broken by reaction with diluted HF to form a cleaved product. A volatile compound is also formed from the cleaved support by reaction with diluted HF.

In addition, at least one of the protecting groups, and preferably all of the protecting groups, remain bonded to the peptide product when the support-linked, protected peptide is cleaved from the support. Most or all of those remaining protecting groups would have been cleaved from the product by reaction with anhydrous HF (e.g., the HF/anisole mixture usually used in the art for such cleavages), had that reagent been used instead of the diluted HF. That diluted HF is thus used to cleave the at least partially protected product from the siliceous support and to convert the siliceous support into the volatile compound(s), but is not used to completely deprotect the product.

It is further noted that a labile group or moiety that can be a protecting group in a given environment can be a desired substituent in another environment. Although that circumstance can exist as where a benzyl ester is present in the desired product of Example 5, that group or moiety is still referred to herein as a protecting group for ease of description.

It is preferred that the reaction product be cleaved from the siliceous support in a single step. Where typically anhydrous HF is used under high acidity conditions (alone or 90 percent in anisole) along with a silica support in peptide synthesis, for example, as in U.S. Pat. No. 6,476,191, the addition of undiluted HF (high concentrations of anhydrous HF in anisole, or anhydrous HF condensed into liquid form) to a side chain-protected support-linked peptide effects complete deprotection, cleavage of the peptide from the support and conversion of the spent silica support into the volatile compound SiF₄ all in one step, although several different reactions are carried out in that one step. It is contemplated that side chain deprotection be carried out separately, as where trifluoroacteic acid is used for that reaction, so anhydrous HF is not used for cleavage and volatilization of the support herein.

The diluent for the HF is preferably a liquid under the conditions of use and is most preferably a liquid at one atmosphere and room temperature. While not wishing to be bound by theory, it is believed that the diluent lowers the acidity of the HF. Thus, anhydrous HF has a pH of −11, whereas a diluted HF composition useful herein for cleavage of the protected product from the volatilizable support and for volatilization of the support has a pH of about zero to about 11, and preferably about 3 to about 8

Most preferably, water is the diluent. An aqueous HF solution is most preferably utilized as a reagent for cleavage of the at least partially protected product from the support, permitting at least one of the protecting groups of the product remain intact after cleavage where that same group would have bee cleaved had anhydrous HF been used for the cleavage reaction. For this purpose, the desired concentrations of HF are in the range of about 5 percent to about 50 percent HF in water. The most preferred concentration of HF is about 10 percent HF in water. Aqueous HF (pH 3-4.5) is notably safer, and more convenient to work with than anhydrous HF because aside from plastic ware, no specialized equipment or containers are required and because it is readily removed by vacuum treatment.

Additionally, other co-solvents can be added to the aqueous HF or used alone with HF to form cleavage reagents that also maintain the integrity of the protecting groups on the product yet still effect volatilization of the support. Illustrative co-solvents include a C₁-C₄ alcohol such as methanol, ethanol, iso-propanol and t-butanol, C₄-C₈ ethers including anisole, diethyl, ethyl propyl, dioxane, tetrahydrofuran (THF), C₁-C₆ amines such as pyridine, dimethylamine, trimethylamine, dimethylsulfide [Tam et al. (1983) J. Am. Chem. Soc., 105:6442-6455 and the citations therein], and mixtures thereof.

It is also contemplated that cleavage of the reaction product from the siliceous support be carried out as a separate step as by the use of triethylamine and methanol, followed by reaction with diluted HF to form the cleaved product peptide and SiF₄ that is then removed by volatilization.

The cleaved, at least partially protected product is preferably recovered directly, and is usually purified by chromatography prior to further use. However, it is also contemplated that the cleaved, at least partially protected product can be further reacted without recovery or further purification.

As used herein, the material formed on the siliceous support and bonded thereto during support-based synthesis is referred to as a “reaction product” or more simply “product”. The reaction product can have at least one protecting group bonded to it in which case it is a “protected product”, or the one or more protecting groups can be absent as where no amino acid side chain protecting were used in the synthesis.

As noted previously, a contemplated improved synthesis can be utilized in the preparation of a number of products such as a peptide (polypeptide), polyamine, peptidomimetic, peptidomimetic amine, glycopeptide, oligonucleotide, or heterocyclic product compound. The terms “peptide”, “polypeptide”, “glycopeptide”, “oligonucleotide” and “oligosaccharide” are sufficiently well known in the biochemical arts to not require further definition.

An oligoamine or polyamine derived from a peptide or “peptidomimetic” can be viewed as an oligo-amine (polyamine) such as an oligopeptide (polypeptide) compound whose amido groups are reduced to amino groups that can be alkylated or not as desired. Illustrative conventional solid phase support-assisted syntheses of peptidomimetic amine compounds are described in U.S. Pat. No. 5,480,971 and No. 6,197,529. An illustrative polyamine synthesis is shown in the Examples that follow.

A “heterocyclic” product compound should also be well known to workers in the biochemical arts. These compounds contain at least ring structure that typically contains three to about eight members, at least one of which is an atom other than carbon: i.e., a “heteroatom”. Usual heterocycles contain one to three rings and one to four heteroatoms such as nitrogen, oxygen or sulfur that is other than carbon. The heteroatoms present can be the same atom as in dioxane, imidazole or purine, or different atoms as in thiazole, oxazole or benzoxazole. Illustrative conventional solid phase support-assisted syntheses of heterocycles are shown in U.S. Pat. No. 6,441,172 and No. 6,545,032.

A “protecting group” is a selectively removable moiety that is used to prevent the reaction of one functional group while another functional group reacts. These moieties are selectively removable in that they can be removed while other protecting or other functionalities do not react. As noted before, a “protecting group” can also be a labile functional group or moiety that one desires to retain as part of the product, but may nonetheless be selectively removable. Protecting groups are well known in the chemical and biological arts and include the t-BOC and Fmoc groups that are used to prevent reaction of amino-terminal amine groups of peptides, the various trityl and substituted trityl groups used in nucleotide chemistry and the acyl and benzyl groups used in protecting saccharidal hydroxyl groups.

More specifically, the term “amino-protecting group” refers to one or more selectively removable substituents on the amino group commonly employed to block or protect the amino functionality. The term “protected (monosubstituted)amino” means there is an amino-protecting group on the monosubstitutedamino nitrogen atom. In addition, the term “protected carboxamide” means there is an amino-protecting group present replacing the proton of the amido nitrogen so that di-N-alkylation cannot occur. Thus, the solid phase support can be deemed to be a protecting group for the C-terminal carboxyl group of a polypeptide when that polypeptide is bonded through a carboxamido nitrogen (actually HN—) to the solid phase support.

Examples of such amino-protecting groups include the formyl (“For”) group, the trityl group (Trt), the phthalimido group, the trichloroacetyl group, Urethane blocking groups, such as t-butoxy-carbonyl (“Boc”), 2-(4-biphenylyl)propyl(2)-oxycarbonyl (“Bpoc”), 2-phenylpropyl(2)oxycarbonyl (“Poc”), 2-(4-xenyl)-isopropoxycarbonyl, 1,1-diphenylethyl-(1)oxycarbonyl, 1,1-diphenylpropyl(1)oxycarbonyl, 2-(3,5-dimethoxyphenyl)propyl(2)oxycarbonyl (“Ddz”), 2-(p-5-toluyl)propyl(2)oxycarbonyl, cyclopentanyloxycarbonyl, 1-methylcyclopentanyl-oxycarbonyl, cyclohexanyl-oxycarbonyl, 1-methylcyclohexanyl-oxycarbonyl, 2-methylcyclohexanyl-oxycarbonyl, 2-(4-toluylsulfonyl)ethoxycarbonyl, 2-(methylsulfonyl)ethoxycarbonyl, 2-(triphenyl-phosphino)ethoxycarbonyl, 9-fluoroenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benz-isoxalylmethoxycarbonyl, 4-acetoxybenzyloxycarbonyl, 2,2,2-trichloro-ethoxycarbonyl, 2-ethynyl(2)propoxycarbonyl, cyclopropylmethoxycarbonyl, isobornyloxycarbonyl, l-piperidyloxycarbonyl, benzyloxycarbonyl (“Z”), 4-phenylbenzyloxycarbonyl, 2-methylbenzyloxycarbonyl, α-2,4,5,-tetramethylbenzyloxycarbonyl (“Tmz”), 4-methoxybenzyl-oxycarbonyl, 4-fluorobenzyloxy-carbonyl, 4-chloro-benzyloxycarbonyl, 3-chloro-benzyloxycarbonyl, 2-chlorobenzyloxycarbonyl, dichlorobenzyloxycarbonyl, 4-bromobenzyloxycarbonyl, 3-bromobenzyloxycarbonyl, 4-nitrobenzyloxycarbonyl, 4-cyanobenzyIoxycarbonyl, 4-(decyloxy)benzyloxycarbonyl, and the like, the benzoylmethylsulfonyl group, dithiasuccinoyl (“Dts’) group, the 2-(nitro)phenylsulfenyl group (“Nps’), the diphenylphosphine oxide group, and like amino-protecting groups. The species of amino-protecting group employed is usually not critical so long as the derivatized amino group is stable to the conditions of the subsequent reactions and can be removed at the appropriate point without disrupting the remainder of the compound. Preferred amino-protecting groups are Boc and Fmoc.

Further examples of amino-protecting groups embraced to by the above term are well known in organic synthesis and the peptide art and are described by, for example T. W. Greene and P. G. M. Wuts, Protective Groups in Organic Synthesis, 2^{nd} ed., John Wiley and Sons, New York, Chapter 7, 1991; M. Bodanzsky, Principles of Peptide Synthesis, 1^{st} and 2^{nd} revised eds., Springer-Verlag, New York, 1984 and 1993; and Stewart and Young, Solid Phase Peptide Synthesis, 2^{nd} ed., Pierce Chemical Co, Rockford. Ill. 1984.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylene-dioxybenzyl, benzhydryl, 4,4′-methoxytrityl, 4,4′,4″-trimethoxytrityl, 2-phenylprop-2-yl, trimethylsilyl, t-butyldimethylsilyl, 2,2,2-trichloroethyl, β-(trimethylsilyl)ethyl, β-[di(n-butyl)methylsilyl]-ethyl, p-toluenesulfonylethyl, 4-nitrobenzylsulfonylethyl, allyl, cinnamyl, 1-(trimethylsilylmethyl)-prop-1-en-3-yl, and like moieties. The species of carboxy-protecting group employed is also usually not critical so long as the derivatized carboxylic acid is stable to the conditions of subsequent reactions and can be removed at the appropriate point without disrupting the remainder of the molecule.

Further examples of these groups are found in E. Haslam, Protective Groups in Organic Chemistry, J. G. W. McOmie Ed., Plenum Press, New York 1973, Chapter 5 and T. W. Greene and P. G. M. Wuts, Protective Groups in Organic Synthesis 2^{nd} ed., John Wiley and Sons, New York, 1991, Chapter 5. A related term is “protected-carboxy”, which refers to a carboxy group substituted with one of the above carboxy-protecting groups.

The term “hydroxy-protecting group” refers to readily cleavable groups bonded to hydroxyl groups, such as the tetrahydropyranyl, 2-methoxyprop-2-yl, 1-ethoxyeth-1-yl, methoxymethyl, β-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl and 2,2,2-trichloroethoxycarbonyl groups, and the like. The species of hydroxy-protecting groups is also usually not critical so long as the derivatized hydroxyl group is stable to the conditions of subsequent reaction(s) and can be removed at the appropriate point without disrupting the remainder of the compound.

Further examples of hydroxy-protecting groups are described by C. B. Reese and E Haslam, Protective Groups in Organic Chemistry, J. G. W. McOmie, Ed., Plenun Press, New York 1973, Chapters 3 and 4, respectively, and T. W. Greene and P. G. M. Wuts, Protective Groups in Organic Synthesis, 2^{nd} ed., John Wiley and Sons, New York, 1991, Chapters 2 and 3.

The “cleaved product” is that material obtained upon breaking of the bond between the support and the reaction product. The cleaved product preferably includes at least one protecting group that would have been cleaved by reaction with anhydrous HF. Regardless of whether that at least one protecting group is present or not, the product is formed using diluted HF under conditions in which the use of anhydrous HF would have cleaved all of the protecting groups. In addition, the cleaved product is typically protonated, although protonation is not a defining feature of a cleaved product.

A “spent support” is the material remaining after cleavage of the desired reaction product from the support. As discussed below, the support is converted into a volatile compound concomitantly with formation of the cleaved product. In that case, there is usually no spent support.

The contemplated support useful herein is a siliceous support that contains silicon, and preferably, each silicon atom is bonded to an average of about two or more oxygen atoms. Thus, materials based on room temperature solid silica (SiO₂) such as glass, as discussed below, and oligo- and polysiloxanes that contain a repeating group -(R¹R²SiO₂)— that are liquids at a temperature of about −70° to about 260° C., and preferably at temperature at which the HF diluent is a liquid, and one atmosphere of pressure are also contemplated supports, wherein R¹ and R² are the same or different and are C₁-C₁₀ alkyl, aryl or aralkyl such as methyl, butyl, or decyl, or phenyl or naphthyl, benzyl or phenethyl, respectively.

The word “glass” is used herein to mean a silica-based solid phase material. Exemplary glass materials include silica glass itself, as well as quartz, borosilicate and aluminosilicate glasses. Still further illustrative glasses are listed on pages 1379-1384 of Van Nostrand's Scientific Encyclopedia, 6^{th} ed. Vol. 1 (1983).

The siliceous support is bonded directly or through a linker, as discussed hereinafter, to the product or protected product. In typical and presently preferred embodiments, all or substantially all of the mass of the support is siliceous and can be volatilized upon treatment with diluted HF.

However, in some embodiments, a silica gel support with its linked, protected product can be utilized to form a matrix of polystyrene or other polymer in situ. Thus, styrene and one or more requisite cross-linking agents can be intercalated into the silica gel, polymerized and the silica gel volatilized to yield a polystyrene matrix that mimics the interstices of the original silica gel and contains the product or protected product of synthesis on the silica support. Stated another way, the solid siliceous support can be utilized as a porous matrix such that upon treatment with the diluted HF, the siliceous support is volatilized away, leaving the product or protected product in the pores of the polystyrene bead.

The siliceous support used in any given synthesis can be in substantially any physical form including without limitation, sheet, tube, fiber and particulate. For example, a sheet of glass such as a piece of plate glass can be prepared to contain linking groups, as discussed hereinafter, and those linking groups can be arrayed in a known manner across the sheet so that syntheses are performed a various, typically predetermined, places on the sheet. Porous glass particles have been used as a support to prepare a peptide with cleavage of the desired product effected by reaction of a solid phase-bound peptide with methanol and triethylamine that provides a spent support and product. [Parr et al., 1974, Justus Liebigs Ann. Chem., pages 655-666.] Contrarily, using a contemplated method, the porous glass can be completely transformed by dilute aqueous hydrogen fluoride into volatile silicon tetrafluoride (SiF₄, bp: −86° C.) that, as necessary, can be warmed or a vacuum applied to effect separation, leaving a product and no spent support. This method can be compared to use of a reagent that cleaves the compound from the support followed by filtration of the spent support from the desired compound as was carried out by Parr et al. Use of a contemplated method leaves the desired compound in the reaction container, with the porous glass support volatilized away as SiF₄.

This support volatilization concept greatly facilitates the production of individual compounds or mixtures of compounds, or the large scale production of individual compounds, arrays of compounds, or combinatorial libraries of mixtures [Plunkett et al., 1995, J. Org. Chem., 60:6006-6007; Houghten, 1985, Proc. Natl. Acad. Sci. USA, 82:5131-5135; Houghten et al., 1991, Nature, 354:84-86; Pinilla et al., 1992, BioTechniques 13:901-905; Ostresh et al., 1994, Proc. Natl. Acad. Sci. USA, 91:11138-11142; Dooley et al., 1994, Science, 266:2019-2022; Eichler et al., 1995, Molecular Medicine Today 1:174-180; and Houghten et al, 1999, J. Med. Chem. 42:3743-3778]. In addition, when working with mixtures of compounds, the risk of losing part of the compounds during the separation process of the support (filtration or centrifugation) is minimized. As noted before, a “protecting group” can also be a labile functional group or moiety that one desires to retain as part of the product, but may nonetheless be selectively removable.

The present invention also contemplates use of a siliceous support that is a polymeric silicone oil support that is liquid at room temperature and one atmosphere of pressure. Polymeric silicone oil supports can be completely volatilized in a manner similar to silica gels, glass or the other previously discussed solid siliceous supports. These oils are typically inexpensive and readily available from Gelest, Philadelphia, Pa. Table 1 (in Example 10, hereinafter) and the reaction Schemes 1-4, below, show several siliceous polymers (silicone oils) of interest and illustrates the results of treating these oils with 100 percent anhydrous HF and 35 percent aqueous HF. As can be seen from reaction Schemes 1-4, in each case, the oils break down to their expected products when exposed to aqueous or anhydrous hydrogen fluoride.

Thus, when 100 mgs of simple methylsiloxanedimethylsiloxane polymer (Scheme 1) was treated with aqueous or anhydrous HF (24 hours and 1.0 hour at room temperature, respectively) no residual weight remained, with all of the silicon oil entirely converted to trimethylfluorosilane (bp=2° C.), dimethyldifluorosilane (bp=16°-18° C.) and water. 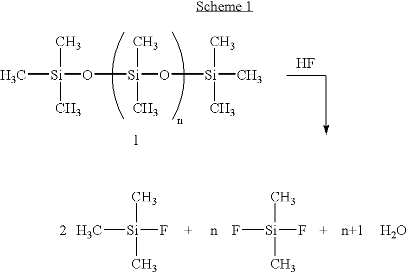
The diphenyl form of the co-polymer (Scheme 2) was also completely volatized following conversion to dimethyldiflurosilanes and benzene (bp=80° C.), and wherein “m” and “n” are average values of repeating unit shown that sum to achieve the average molecular weight shown in Table 1 for a siliceous oil of Schemes 1-4.

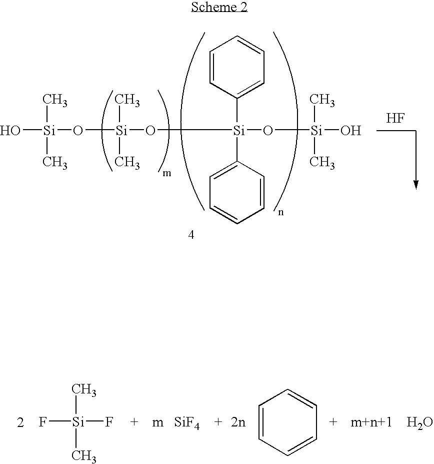

For the mono- and di aminopropyl functionalized copolymers shown in Schemes 3 and 4, below, and in Table 1, the weights remaining following treatment of 1000 mgs of each corresponded exactly with that expected following the volatilization of the silyl components with the residual single or double aminopropylmethyldifluorosilane (Table 1) left as residual materials.

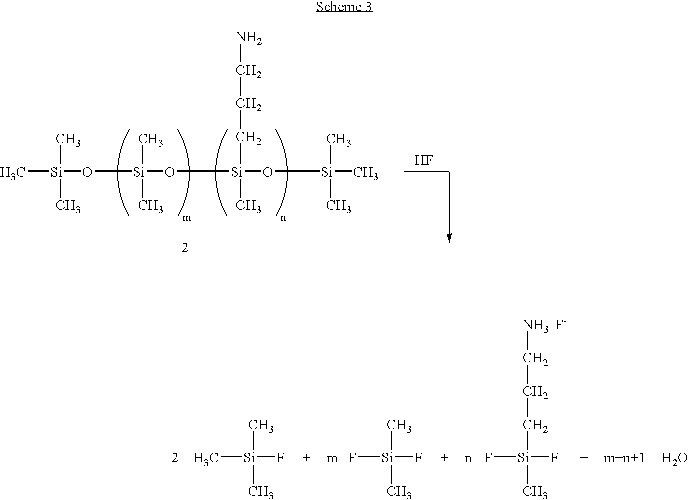

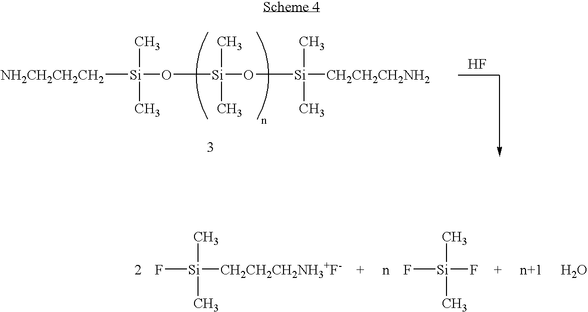

These silicone oils are quite insoluble in water and quite soluble in toluene, THF and dichloromethane. These oils can thus serve as soluble polymeric supports for organic synthesis in a manner similar to that pioneered by Janda and co-workers [Gravert et al., 1997 , Chemical Reviews 97:489-510]. Along with the advantage of enabling their complete volatilization following the synthesis of specific compounds, the support-bound materials can be readily studied by proton NMR because the methylsilyl polymeric groups are seen below 0.5 PPM, an area typically free of signals. Oils that are per-fluorinated can also be used and exhibit no signals in the region typically seem for H-NMR.

The present invention also contemplates the use of so-called non-cleavable linkers in connection with such volatilizable supports. A non-cleavable linker is a linker that remains bonded to the cleaved product, but is cleaved from the support. This use leads, after cleavage, to a modified compound (compound attached to linker) that can be of interest in itself, or that can be further modified if necessary.

Exemplary non-cleavable linkers can be prepared using amino-C₂-C₆-alkyl-grafted glass beads as a solid support to prepare a compound such as a peptide. Exemplary aminopropyl glass beads having different pore sizes, mesh sizes and micromoles of primary amine per gram of glass (μmol/g) are commercially available from Sigma Chemical Co., St. Louis, Mo., as is aminopropyl silica gel that is said to contain nitrogen at 1-2 mmoles/g.

Thus, use of aminopropyl-grafted glass beads to form the siliceous support-linked, protected peptide, followed by treatment with diluted HF provides a protected peptide with a C-terminal trifluorosilylpropylamido (—CO—NH—CH₂—CH₂—CH₂—SiF₃) group that can be readily hydrolyzed to form the corresponding silicic acid group [—CO—NH—CH₂—CH₂—CH₂—Si(OH)₃]. This compound, after partial or complete polymerization through the —Si(OH)₃ group, can be used as a conjugate for immunization in the preparation of antibodies against the peptide of interest. Furthermore, such materials can be useful for the affinity purification of polyclonal antibodies generated against the peptide or the compound of interest. The silicon atom can also be present after such hydrolyses as a —Si(OH)₂F or —Si(OH)F₂ group, which can also be used in a polymerization or other reaction. Alternatively, oxidation with 30 percent hydrogen peroxide in water cleaves the carbon-silicon bond to form a hydroxypropylamido- (—CO—NH—CH₂—CH₂—CH₂—OH) terminated peptide, and separates the product from the support, so that subsequent treatment with diluted HF provides a volatilizable silicon compound that can be separated from the product hydroxypropylamido-terminated peptide under reduced pressure.

In addition to an aminopropyl group, other linking groups are also contemplated. For example, 3-mercaptopropyltrimethoxysilane [HS—CH₂—CH₂—CH₂—Si(OCH₃)₃] available from Hüls America, Inc., Piscataway, N.J. can be coupled to porous glass beads to provide 3-mercaptopropyl-grafted glass (thiolated glass). Reaction of the thiolated glass with bis-N-BOC-2-aminoethyl disulfide provides a primary amine-terminated disulfide after deprotection. The primary amine can be used to synthesize peptides in a usual solid phase synthesis. Upon completion of the synthesis, treatment of the reaction product-linked glass with a reducing agent and then aqueous HF provides a protected peptide having a C-terminal amidoethylmercapto group and a vaporizable remnant of the support. The amidoethylmercapto-terminated protected peptide can be readily reacted with an antigenic carrier molecule previously reacted with m-maleimidobenzoyl-N-hydoxysuccinimide ester (ICN Biochemicals, Inc., Costa Mesa, Calif.) or succinimidyl 4-(N-maleimidomethyl)cyclohexane-1-carboxylate (SMCC, Pierce Chemical Co., Rockford, Ill.) to form an immunogenic conjugate. Further useful groups for linking immunogenic materials to carrier molecules can be found in the Pierce Chemical Co. catalog.

The disulfide-containing BOC-protected linking group precursor can be prepared by standard techniques. For example, 2-aminoethyl disulfide can be reacted with two moles of 2-(tert-butoxycarbonyloxylmino)-2-phenylacetonitrile or N-(tert-butoxycarbonyloxy)phthalimide or a similar reagent to form bis-N-BOC-2-aminoethyl disulfide.

Several reducing reagents are well known to be useful for breaking the disulfide bond. Exemplary reagents include sodium borohydride, 2-mercaptoethanol, 2-mercaptoethylamine, dithiothreitol and dithioerythritol. Mercaptan-containing carboxylic acids having two to three carbon atoms and their alkali metal and ammonium salts are also useful. Those reagents include thioglycolic acid, thiolactic acid and 3-mercaptopropionic acid. Exemplary salts include sodium thioglycolate, potassium thiolactate, ammonium 3-mercaptopropionate and (2-hydroxyethyl)ammonium thioglycolate.

The use of cleavable linking groups that separate both from the cleaved product and from the support is also contemplated. One group of cleavable linkers contains a benzyl group and silicon. Upon treatment with specific reagents like dilute HF, such cleavable linkers can be transformed into gases or liquid forms that can be readily volatilized at various useful temperatures and pressures. Such linking groups are thus cleavable and form volatile compound(s) on reaction of HF with the support.

For example, linkers such as Cl—CH₂C₆H₄—(CH₂)₃₋₅—SiCl₃, Cl—CH₂C₆H₄—(CH₂)₃₋₅—Si(CH₃)Cl₂, Cl—CH₂C₆H₄—(CH₂)₃₋₅—Si(CH₃)₂Cl, Cl—CH₂C₆H₄—SiCl₃ and Cl—CH₂—C₆H₄—Si(OCH₃)₃ can be reacted with glass beads (or any SiO₂-based or other siliceous material) to form α-chlorobenzyl C₃-C₅-alkyl-grafted glass beads or α-chlorobenzyl-grafted glass beads, respectively, that contain one or more siloxane bonds with the support. Exemplary α-cholorbenzyl C₃-C₅-alkyl chlorosilanes and α-chlorobenzyl chloro- or methoxysilanes are available from Hüls America, Inc., Piscataway, N.J. This grafted glass support can thereafter be reacted through the chloromethyl group with a wide variety of compounds such as protected amino acids, amines, alcohols, and the like to form benzyl ether groups. In the case where n=1 and one methylene group is present between the ring and silicon atom, this linker can be transformed into the volatile para(trifluorosilylmethyl)benzyl fluoride (F—CH₂C₆H₄—CH₂—SiF₃) by treatment with a solution of hydrogen fluoride as discussed hereinafter.

It is also contemplated as a part of this invention to use what is termed a “non-traceless” linker between the support and the product. A traceless linker [Plunkett and Ellman, 1995, J. Org. Chem. 60:6006-6007] is completely removed during the cleavage/volatilization reactions, and is exemplified by hydroxymethylphenyl silyl ethers and esters and aminomethylphenyl silyl linkers. On the other hand, non-traceless linkers remain attached to the product. Here in one aspect, a two step process is utilized in which an alkylsilyl group is cleaved to form the corresponding product-linked alkylhydroxyl group and a spent silyl support. For example, an aminopropylsilica-linked peptide is treated with aqueous hydrogen peroxide to form a 3-hydroxypropylamidopeptide and silica. Treatment of that reaction mixture with 10 percent HF in water provides volatile silicon-containing products and the desired hydroxypropylamidopeptide product.

The following Examples are offered to further illustrate, but not limit the present invention.

### EXAMPLE 1

### Completeness of Volatilization of Silica Gel: Anhydrous HF vs. Aqueous HF

Silica gel samples (1.0 g, Gelest™ and/or Silicycle™, Sigma-Aldrich) were treated with either anhydrous HF (4.0 ml) or aqueous HF (4.0 ml) in concentrations that ranged from 5-50 percent HF for one hour at room temperature. The residue was lyophilized then weighed and determined to be about 5 mg in all cases.

### EXAMPLE 2

### Volatilization of Functionalized Silica Gel: Anhydrous vs. Aqueous HF

p-Chloromethylphenyl silica gel [1.0 milliequivalents per gram (meq/g), 1.0 g, Silicycle™] was treated with either anhydrous HF (4.0 ml), 90 percent HF in anisole (4.0 ml), or 10 percent HF in water (4.0 ml) for one hour at 4° C. The products were examined by ultra-violet spectroscopy. The product of the anhydrous HF reaction yielded about 5 mg of UV visible products, the product of the HF/anisole reaction yielded about 50 percent less UV visible products, and the product of the 10 percent HF/water reaction had very little UV visible products, thereby indicating the most complete conversion and volatilization of the solid support.

### EXAMPLE 3

### Solid-phase Peptide Synthesis and Volatilization of Functionalized Silica Gel in Aqueous HF

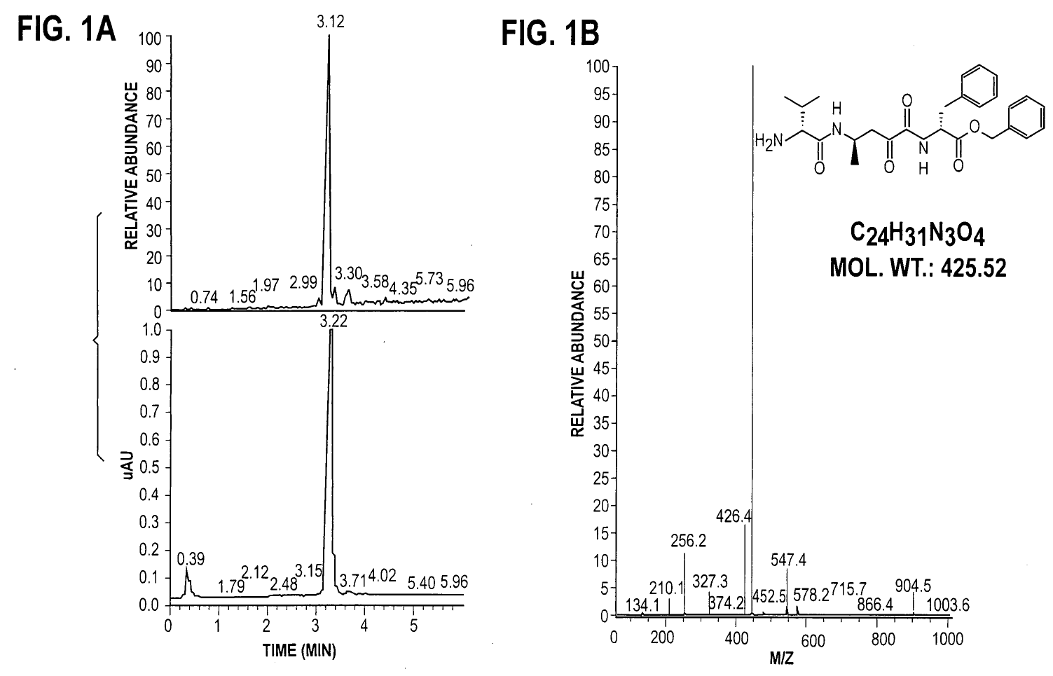

The C-terminal benzyl ester of L-valine-L-alanine-L-phenylalanine was prepared on phenylmethylchloro silica gel (1.0 meq/g, 1.0 g, Silicycle™) using standard Boc peptide synthesis chemistry (Boc/TFA/diisopropylcarbodiimide (see for example, A. Nefzi et al., 1999 Tetrahedron 55:335-344). Following removal of the N-terminal Boc group with TFA, the silica gel-benzyl ester linked peptide was treated with 10 percent HF in water (4.0 ml) at room temperature for one hour. The product was lyophilized. The crude yield was 0.43 g or about 95 percent based on the weight of the starting material. The purity of the product peptide is illustrated by the HPLC-MS (M+Na) shown in FIG. 1. The benzyl ester would have been cleaved by reaction in anhydrous HF.

### EXAMPLE 4

### Stability of Saccharides Under Volatilization Conditions Using 10% HF in Water

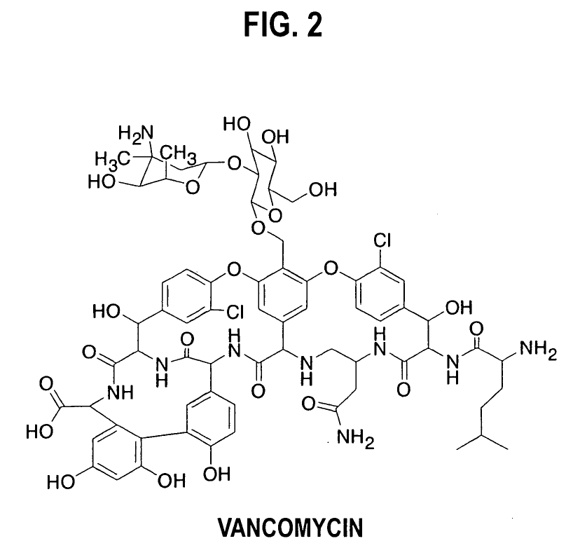

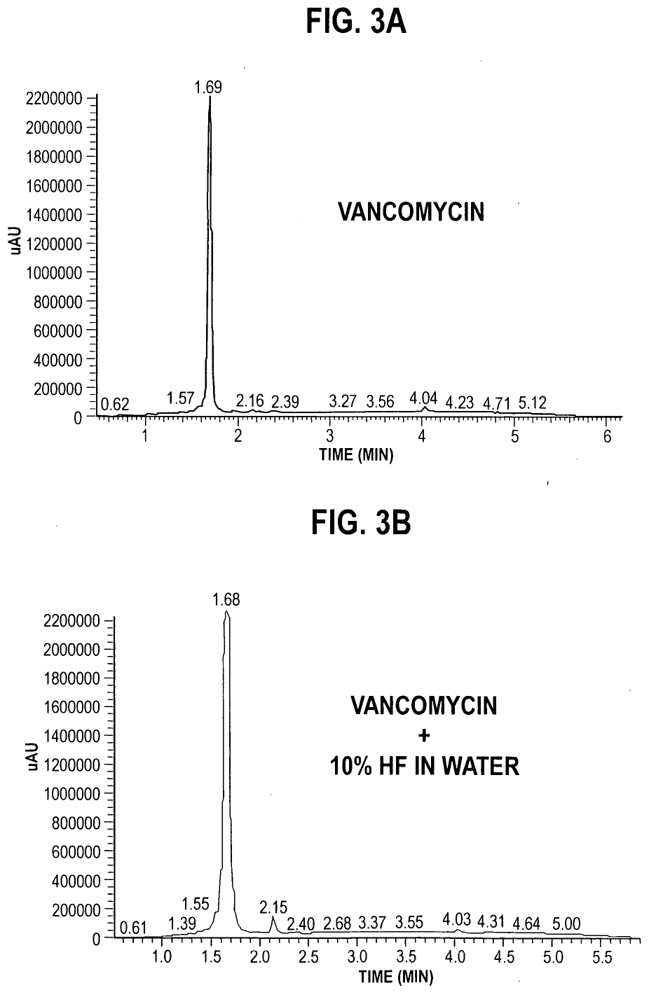

Vancomycin, an oligosaccharide mimic (FIG. 2), (0.10 g) was treated with 10 percent HF in water (4.0 ml) at room temperature for overnight (about eighteen hours). The stability of Vancomycin was greater than 95 percent as illustrated by the HPLC chart shown in FIG. 3.

### EXAMPLE 5

### Solid Phase Synthesis of Heterocyclics and Peptidomimetics

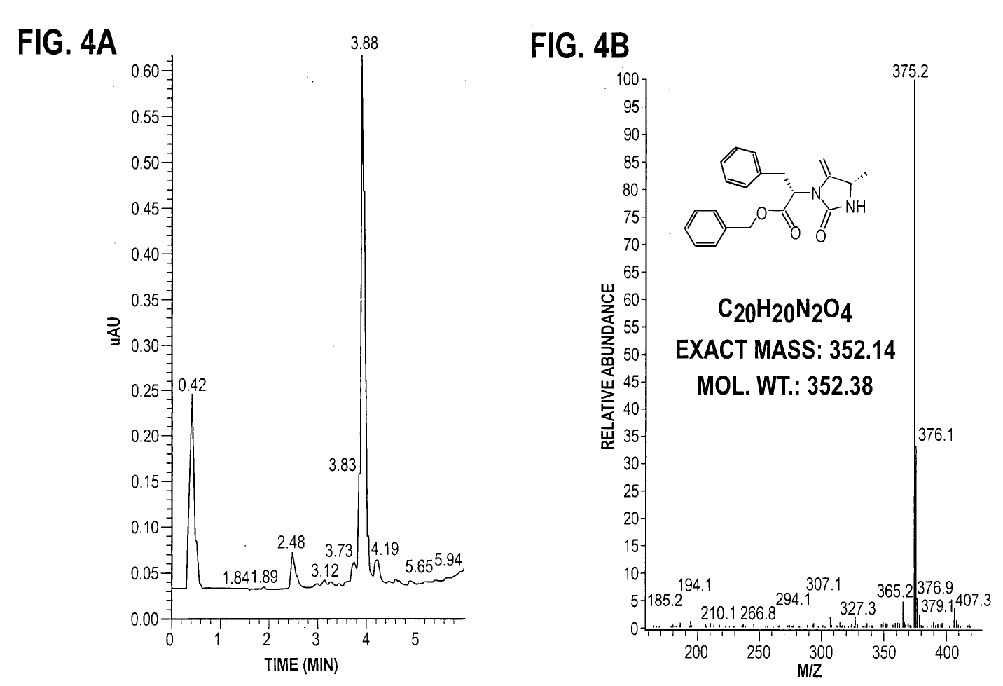

A simple peptidomimetic was prepared. This hydantoin O-benzyl ester (FIG. 4), was obtained from the treatment of silica gel-bound O-benzyl ester of N-phenylacetyl-L-alanine with carbonyldiimidazole. (Nefzi et al. 1997 Tetrahedron Lett. 38:931-934). The recovered yield using 10% HF in water to cleave the protected product from the support, RP-HPLC, mass spectral analysis and NMR of this compound were completely in line with the expected hydantoin benzyl ester.

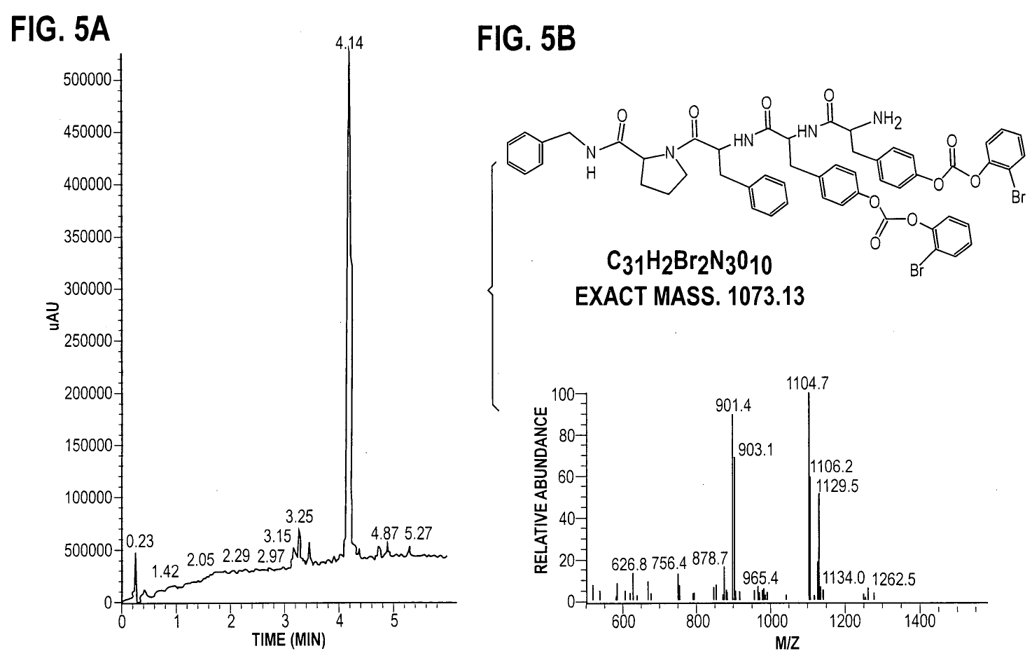

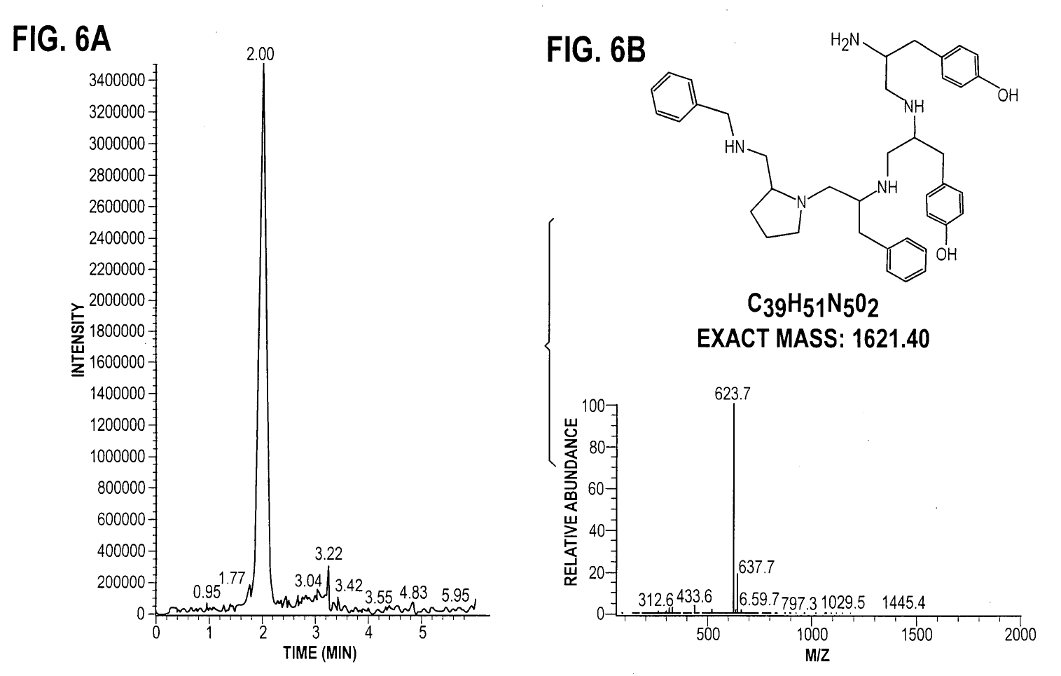

In another preliminary study, a p-benzylamine silica gel-bound L-tyrosine(BrZ)-L-tyrosine(BrZ)-L-phenylalanine-L-proline prepared on phenylmethylamine silica gel using standard Boc peptide synthesis chemistry (Boc/TFA/diisopropylcarbodiimide. Following removal of the N-terminal Boc group with TFA, the silica gel-benzyl ester linked peptide was treated with 10 percent HF in water. The product was lyophilized. The crude of the product peptide is illustrated by the HPLC-MS (M+Na) shown in FIG. 5. The p-benzylamine amide silica gel-bound L-tyrosine(BrZ)-L-tyrosine(BrZ)-L-phenylalanine-L-proline as shown in FIG. 5B could be reduced to a chiral polyamine. Under the conditions examined, the desired polyamine was obtained in a purity of approximately 75 percent. Here, the product of the reduction on silica-support of the L-tyrosine(BrZ)-L-tyrosine(BrZ)-L-phenylalanine-L-proline N-benzylamine is shown in FIG. 6. The reduced polyamine can be used to prepare a wide variety of heterocyclic compounds. [Nefzi et al., 2001, Biopolymers 60:212-219; Blaney et al., 2002, Chem Rev. 102:2607-2624; and Parr et al., 1971, Tetrahedron Lett. 12:2633-2636.]

### EXAMPLE 6

### Solid-phase Synthesis of 1,6-Disubstituted 2,3-Diketopiperazines

A series of 1,6-disubstituted 2,3-diketopiperazines of the structural formula below are prepared following the general procedures described in Nefzi et al., 1999, Tetrahedron Lett. 40:8539-8542, except that the R²-containing amido-protected compounds are removed from a silica-based solid support using 10% HF in water, which results in the formation of volatilizable silica compounds that are removed under reduced pressure. The R² group in these compounds is the residuum of a C₁-C₂₀ carboxylic acid, whereas the R¹ group is an amino acid side chain that can contain a protecting group.

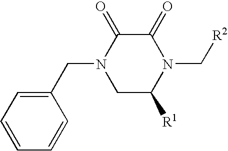

Thus, following Boc deprotection and neutralization from an p-aminomethylphenylsilica-bound Boc protected amino acid, the free amine is N-acylated with a variety of commercially available carboxylic acids in the presence of diisopropylcarbodiimide (DIPCDI) and hydroxybenzotriazole (HOBt). The amide bonds are then reduced to generate two secondary amines that, following treatment with oxalyldiimidazole and 10% hydrogen fluoride in water cleavage, provide the desired diketopiperazines and the solid support in volatilizable form.

### EXAMPLE 7

### Solid Phase Preparation of 1,4-Benzothiazepin-5-one Compounds

A series of 1,4-benzothiazepin-5-one compounds shown below wherein R² is as defined above and R¹ is the residuum of a reductively alkylated C₁-C₁₀ aldehyde. These compounds are prepared following the general synthesis procedures of Nefzi et al., 1999, Tetrahedron Lett 40:4939-4942.

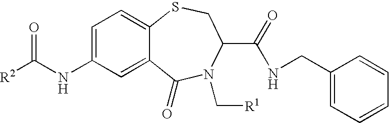

Thus, N-α-Fmoc-S-trityl-L-cysteine is coupled to p-aminomethylphenylsilica in the presence of diisopropylcarbodiimode (DIPCDI) and hydroxybenzotriazole (HOBt). Following cleavage of the trityl (Trt) group with 10% trifluoroacetic acid (TFA) in dichloromethane (DCM) in the presence of 5% of tBu₃SiH, 2-fluoro-5-nitro-benzoic acid is added to the resin-bound Fmoc-cysteine. The Fmoc group is cleaved by reaction with 25% piperidine in DMF, and the resulting free amine is reductively alkylated with a variety of C₁-C₁₀ aldehydes discussed above in the presence of sodium cyanobrorohydride. The resulting resin-bound intermediate is treated with O-benzotriazolyl-N,N,N′,N′-tetramethyluronium hexafluorophosphate (HBTU) in anhydrous DCM, which undergoes an intramolecular amide bond formation to afford a solid phase-bound nitrobenzothiazepine. The nitro group is reduced with SnCl₂, followed by N-acylation using an above-described R²-containing carboxylic acid, and yields the desired product following treatment with 50% HF in water.

### EXAMPLE 8

### Preparation of Reactive Silica Gels

Silica gel, 130-270 mesh, 60 Å, BET surface area 500 m²/g, pore volume 0.75 cm³/g, was purchased from Aldrich Chemical Company, Inc. 100 Grams of that silica gel was refluxed with 100 ml conc. HCl for 6 hours, washed with water until pH=6-7, and dried under vacuum.

A. Preparation of Pure (p-Phthalimidomethyl)phenyltriethoxysilane

Following the reaction shown in Scheme 5, below, a mixture of (p-chloromethyl)phenyltrimethoxysilane (2.47 g, 10 mmol) and phthalimide potassium (2.04 g, 11 mmol) in 30 ml of anhydrous ethanol was stirred at 80° C. for 24 hours. The mixture was filtered and the ethanol was evaporated under reduced pressure. The residue, in which the methoxy groups were exchanged during heating to ethoxy groups, was purified by silica gel column chromatography using Hexane:EtOAc (5:1 v/v) as the eluent, and a white solid was obtained in 78% yield. ¹H NMR (CDCl₃, 500 MHz) δ 1.21 (9H, t, J=7.0), 3.80-3.85 (6H, m), 4.84 (s, 2H), 7.41-7.83 (8H, m)

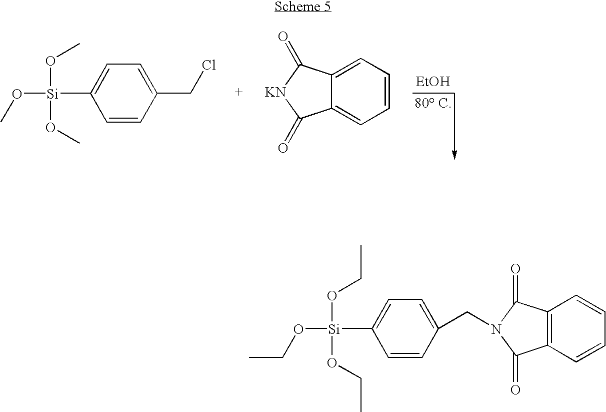

B. Preparation of Crude (p-Phthalimidomethyl)phenyltriethoxysilane

A mixture of (p-chloromethyl)phenyltrimethoxysilane (2.47g, 10 mmol) and potassium phthalimide (2.04 g, 11 mmol) in 30 ml of anhydrous ethanol was stirred at 80° C. for 24 hours. The mixture was filtered, and after the ethanol was removed by evaporation, any residual (p-chloromethyl)phenyltriethoxy silane was removed evaporated under reduced pressure (10 mm Hg) at 160° C., to afford the crude product. The crude (p-phthalimidomethyl)phenyltriethoxy silane was directly used to load on to silica gel without further purification.

C. Preparation of Functionalized Benzylamine Silica Gel, Loading of (P-phthalimidomethyl)phenyltriethoxysilane on Silica Gel

Following the reaction illustrated in Scheme 6, below, wherein the shaded lines indicate the surface of the reacted silica gel, 1.0 g silica gel was sealed within a polypropylene mesh packet. (1.0 g, 2.5 mmol) (p-phthalimidomethyl)phenyltriethoxysilane and 20 ml of anhydrous toluene were added to the silica. The mixture was heated at 100° C. overnight (about 18 hours). The bag was washed with DMF (3 times), DCM (3 times) and dried in air.

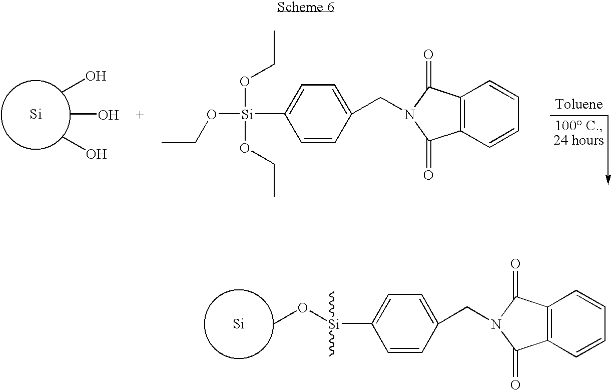

D. Benzylamine Silica Gel Resin

The above 1.0 g silica gel and a solution of 1 ml hydrazine in 20 ml ethanol were heated at 80° C. overnight (about 18 hours) as shown in Scheme 7, below. The silica gel was washed with DMF (3 times), DCM (3 times) and dried in air to provide the corresponding benzylamine silica gel resin (0.1-1.4 mmol/g).

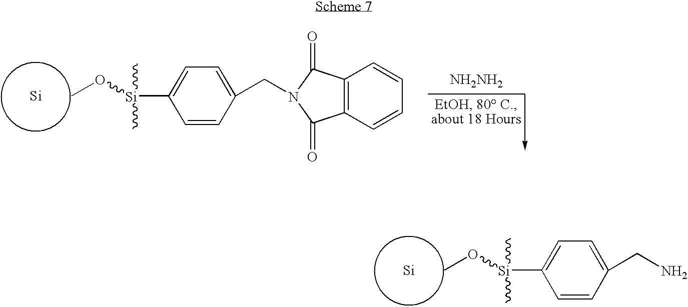

### EXAMPLE 9

### Preparation of Substituted 1,2-Diketopiperazines and Libraries

By analogy to the syntheses disclosed in U.S. Pat. No. 6,441,172, starting from benzylamine silica gel resin discussed above bound to a first fluoroenylmethoxycarbonyl amino acid (Fmoc-R¹aa-OH), the Fmoc group is removed using a mixture of piperidine in dimethylformamide (DMF). The resulting free amine is then protected with triphenylmethyl chloride (TrtCl). The secondary amide is then selectively alkylated in the presence of lithium t-butoxide and alkylating reagent, R²X, in this instance methyl iodide or benzyl bromide to form the resin-bound N-alkylated compound. The Trt group is cleaved with a solution of 2% trifluoroacetic acid (TFA) and a second amino acid (Fmoc-R³aa-OH) was coupled in presence of diisopropylcarbodiimide and hydroxy-benzotriazole, and the Fmoc protecting group is removed to form the resin-bound dipeptide. The resin bound-dipeptide is N-acylated with a wide variety of carboxylic acids (R^{4a}COOH) to form the resin-bound N-acylated dipeptide. Exhaustive reduction of the amide bonds of the resin-bound N-acylated dipeptide is achieved using borane in tetrahydrofuran as described, for instance, in Ostresh et al., 1998, J. Org. Chem., 63:8622-8623 and in Nefzi et al., 1999, Tetrahedron, 55:335-344. The resulting resin-bound polyamine is then treated with oxalyldiimidazole in anhydrous DMF to form resin-bound diketopiperazine. Reaction of that resin-bound compound with 10 percent HF in water provides a desired diketopiperazine, whose structure is shown below, wherein R¹ and R³ are amino acid side chains, R² results from the reduction of N-alkylated amino acid, and R⁴ results from the reduction of the N-acyl group.

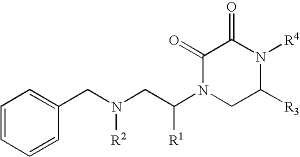

Following the strategy described above, with the parallel synthesis approach, commonly referred to as the “T-bag” method [Houghten et al., 1991, Nature, 354:84-86], with 29 different amino acids at R¹, 27 different amino acids at R³, 40 different carboxylic acids at R⁴, libraries containing 97 different N-benzyl-diketopiperazines, (R²=Bzl) and 97 different N-methyl diketopiperazines, (R²=Me) are synthesized in which the individual building blocks are varied while fixing the remaining two positions.

### EXAMPLE 10

### Preparation of Substituted [3,5,7]-1H-imidazo-[1,5-a]-imidazol-2(3H)-ones and Libraries

By analogy to the syntheses disclosed in U.S. Pat. No. 6,545,032, starting from benzylamine silica gel resin discussed before bound to a first N-tert-butyloxycarbonyl (Boc) amino acid (Boc-R¹aa-OH), the Boc group is removed using 55% trifluoroacetic acid (TFA) in dichloromethane (DCM). The resulting amine salt is neutralized, and the resulting primary amine is N-acylated with a second Boc-protected amino acid (Boc-R²aa-OH) as before, to provide the resin bound-monopeptide.

Following removal of the Boc protecting group using 55% of trifluoroacetic acid in dichloromethane, the resulting free amine is acylated with a carboxylic acid (R³—CO₂H) in dimethylformamide (DMF) using diisopropyl-carbodiimide (DICI) and hydroxybenzotriazole (HOBt) to effect coupling. The bicyclic [3,5,7]-1H-imidazo[1,5-a]-imidazol-2(3H)-one is obtained via cyclization using the conditions of Bischler-Napieralski, with 25-fold excess of phosphorus oxychloride (POCl₃) in refluxing 1,4-dioxane in the presence of a 30-fold excess of anion exchange resin (AG® 3-X4) [Fodor et al., 1981, Heterocycles, 15:165] and the citations therein. Syntheses using freshly distilled POCl₃ in the absence of the anion exchange resin provide yields in the range of about 80 percent. The desired products are readily obtained following cleavage from and volatilization of the silica resin with 10 percent HF in water to provide compound whose structural formula is shown below, wherein R¹ and R² are amino acid side chains and R³ is the residuum of an acylated carboxyl group.

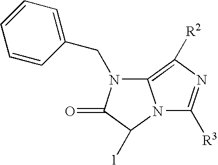

Following the strategy described above, using the “tea-bag” method parallel synthesis approach, [Houghten et al., 1991, Nature, 354:84-86], libraries are synthesized with 33 different amino acids to provide the R group at R¹, 33 different amino acids to provide the R group at R², and 92 different carboxylic acids to provide the R group at R³ as discussed in U.S. Pat. No. 6,545,032, in which the individual building blocks were varied, while fixing the remaining two positions.

Illustrative thirty-three first amino acids can include BOC-protected Gly, His(DNP), Ile, Lys(CBZ), Leu, Met, Arg(Tos), Nva, Ser(Bzl), Thr(Bzl), Val, Tyr(CHO), Tyr(BrZ), Nle, Cha, ala, phe, his(DNP), ile, lys(CBZ), leu, met, arg(Tos), ser(Bzl), thr(Bzl), val, trp(CHO), tyr(BrZ), nle, nva, cha, wherein all lower case designations indicate D amino acids. One of those amino acids is coupled to the silica resin and after removal of the BOC protecting group, the same or different single amino acid of the illustrative 33 is coupled as the second amino acid, thereby providing the R² group. After removal of the second BOC group, a single carboxylic acid, acetic acid, is coupled to provide the R³ group for the 33 different compounds. Those compounds are thereafter cyclized to form compounds of the above structural formula, and then cleaved from and volatilization of the silica resin.

Another set or sub-library of 33 compounds is prepared by reacting a single amino acid [e.g., Tyr(BrZ)] with the resin to provide one R¹ group. After removal of the BOC protecting group, each of the above 33 amino acids is then separately coupled to provide 33 resin-linked peptides with the same R¹ group and one of the 33 different R² groups. On removing the second BOC group, a single carboxylic acid (acetic acid) is bonded to the free amino group to provide a single R³ group for the resin-linked peptides. Theses compounds are also cyclized to form compounds of the above formula, and cleaved from the silica resin with volatilization.

In a third set or sub-library preparation, a single amino acid [e.g., Tyr(BrZ)] is coupled to the resin to provide a single R¹ group, the BOC group is removed and a second amino acid (valine) was coupled to provide a single R² group and form a dipeptide. After removal of the second BOC group, the dipeptide is separately reacted with each of the 92 carboxylic acids listed in Table 2 of U.S. Pat. No. 6,545,032 to provide 92 different R³ groups. The acylated peptides are thereafter cyclized, cleaved from silica resin with volatilization and recovered.

### EXAMPLE 10

### Reaction of Aminosilicone Polymer Oils with Aqueous HF

A series of aminosilicone oil 1000 mg samples were reacted with 35 percent HF in water at room temperature for 24 hours, and further 1000 mg samples of the same oils were reacted with anhydrous HF at 4° C. for one hour. The products of the reaction were volatilized and the residues compared. The results were the same for both treatments and are shown below for the aqueous HF study.

TABLE 1
Reaction treatment of silicone oils with HF^{A}

| | | | Non-volatile | Residue |
|---|---|---|---|---|
| Name^{B} | | Viscosity | Reaction | Actual |
| (Mol Wt) | Structure | (cps) | Product | Theory |
| Silicone Oil | 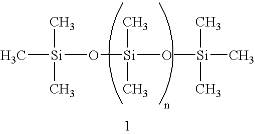 | 80-100 | none | Zero Zero |
| AMS-162 (MW≅4500-5000) | 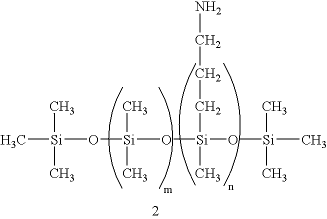 | 80-100 | aminopropyl- difluoro- methylsilane^{C} | 50 mg 58 mg |
| DMS-A11 (MW≅850-900) | 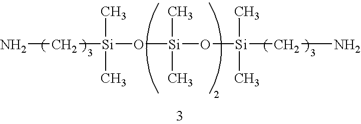 | 10-15 | aminopropyl- fluoro dimethylsilane^{D} | 280 mg 270-320 mg |
| DMS-A21 (MW≅5000) | 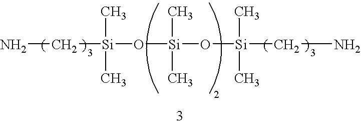 | 100-120 | aminopropyl fluoro dimethylsilane^{D} | 58 mg 51-60 mg |
| PDS-1615 (MW≅900-1000) | 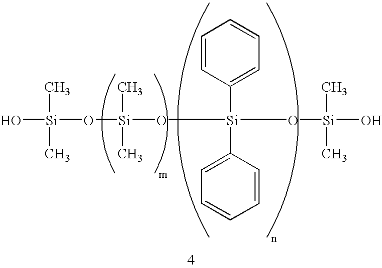 | 50-60 | none | Zero Zero |

^{A}Identical results were obtained with 35% aqueous HF (24 hours, room temperature) and anhydrous HF (1 hour, 4° C.).
^{B}Gelest, Philadelphia, PA; Mol Wt = MW = molecular weight.
^{C}MW of HF amine salt = 159.
^{D}MW of HF amine salt = 155.

Each of the patents and articles cited herein is hereby incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. In a support-based synthesis method wherein at least one reagent having at least one protecting group is coupled to a support to form a protected support-coupled reagent, at least one reaction is carried out upon the protected support-coupled reagent to form a protected support-coupled reaction product and that reaction product is cleaved from the support to form a cleaved product having at least one bonded protecting group, the improvement in which the support is reacted with diluted HF to form a volatile compound that is separated from the cleaved product by vaporization of that formed volatile compound, said reaction with diluted HF being carried out under conditions such that at least one protecting group that would have been cleaved by reaction with anhydrous HF remains bonded to the cleaved product.

2. The support-based synthesis method according to claim 1 wherein said support is siliceous.

3. The support-based synthesis method according to claim 2 wherein said siliceous support is glass.

4. The support-based synthesis method according to claim 2 wherein said siliceous support is benzylamine silica gel resin.

5. The support-based synthesis method according to claim 1 wherein said at least one reagent coupled to said support is an amino acid.

6. The support-based synthesis method according to claim 5 wherein said cleaved product is a peptide.

7. The support-based synthesis method according to claim 5 wherein said cleaved product is a glycopeptide.

8. The support-based synthesis method according to claim 5 wherein said cleaved product is an oligoamine.

9. The support-based synthesis method according to claim 5 wherein said cleaved product is a heterocycle.

10. The support-based synthesis method according to claim 1 wherein said at least one reagent coupled to said support is a saccharide.

11. The support-based synthesis method according to claim 10 wherein said cleaved product is an oligosaccharide.

12. The support-based synthesis method according to claim 1 wherein reaction product is cleaved from said support and the support is reacted with diluted HF to form a volatile compound in a single step.

13. The support-based synthesis method according to claim 12 wherein said single step is carried out by reaction of the support-coupled reaction product with hydrogen fluoride diluted in water.

14. The support-based synthesis method according to claim 1 including the further step of recovering the cleaved product.

15. In a support-based synthesis method wherein at least one reagent with at least one protecting group is coupled to a siliceous support, at least one reaction is carried out upon the protected siliceous support-coupled reagent to form a protected siliceous support-coupled product that is cleaved from the support to form a cleaved product, the improvement in which the support is reacted with diluted HF to form a volatile compound that is separated from the protected cleaved product by vaporization of that formed volatile compound, and said reaction with diluted HF is carried out under conditions such that at least one protecting group that would have been cleaved by reaction with anhydrous HF remains bonded to the cleaved product

16. The support-based synthesis method according to claim 15 wherein said at least one reagent coupled to said siliceous support is an amino acid.

18. The support-based synthesis method according to claim 16 wherein said cleaved product is a peptide.

19. The support-based synthesis method according to claim 15 wherein said at least one reagent coupled to said siliceous support is coupled to said support by means of a linking group.

20. The support-based synthesis method according to claim 19 wherein said linking group is cleavable.

21. The solid phase synthesis method according to claim 19 wherein said siliceous support is reacted α-chlorobenzyl C₃-C₅-alkyl-grafted glass beads.

22. The support-based synthesis method according to claim 19 wherein said linking group is non-cleavable.

22. The support-based synthesis method according to claim 21 wherein said glass support is amino-C₂-C₆-alkyl-grafted glass beads.

23. The support-based synthesis method according to claim 15 wherein said diluted HF has a pH value of about zero to about 11.

24. The support-based synthesis method according to claim 23 wherein said diluted HF has a pH value of about 3 to about 8.

26. The support-based synthesis method according to claim 23 wherein said diluted HF is present at about 5 to about 50 percent in water as diluent.

26. The support-based synthesis method according to claim 15 wherein said siliceous support is comprised of solid particles.

27. The support-based synthesis method according to claim 15 wherein said siliceous support is a liquid a room temperature and one atmosphere of pressure.

28. The support-based synthesis method according to claim 15 wherein said siliceous support is a liquid at room temperature and one atmosphere of pressure.

29. The support-based synthesis method according to claim 28 wherein said siliceous support is an aminosilicone oil.

30. A method for support-based synthesis of a product having at least one protecting group that would have been cleaved by reaction with anhydrous HF comprising the steps of:
(a) coupling at least one reagent having at least one protecting group to a siliceous support to form a protected support-coupled reagent;
(b) reacting the protected support-coupled reagent with at least one reagent having at least one protecting group to form a protected support-coupled product; and
(c) cleaving the protected support-coupled product from the support to form a protected cleaved product by reaction with diluted HF, said reaction with diluted HF being carried out under conditions such that at least one protecting group that would have been cleaved by reaction with anhydrous HF remains bonded to the cleaved product.

31. The support-based synthesis method according to claim 30 wherein said siliceous support is particulate.

32. The support-based synthesis method according to claim 30 wherein said siliceous support is a liquid at a temperature of about −70° C. to about 260° C. and one atmosphere of pressure.

33. The support-based synthesis method according to claim 30 wherein said at least one reagent coupled to said support is an amino acid.

34. The solid phase synthesis method according to claim 33 wherein said cleaved product is a peptide.

35. The support-based synthesis method according to claim 33 wherein said cleaved product is a glycopeptide.

36. The support-based synthesis method according to claim 33 wherein said cleaved product is an oligoamine.

37. The solid phase synthesis method according to claim 33 wherein said cleaved product is a heterocycle.

38. The solid phase synthesis method according to claim 30 wherein said at least one reagent coupled to said support is a saccharide.

39. The solid phase synthesis method according to claim 38 wherein said cleaved product is an oligosaccharide.

40. The solid phase synthesis method according to claim 30 wherein reaction product is cleaved from said support and the support is reacted to form a volatile compound in a single step.

41. The solid phase synthesis method according to claim 40 wherein said single step is carried out by reaction of the support-coupled reaction product with hydrogen fluoride diluted with water.

42. The solid phase synthesis method according to claim 30 including the further step of recovering the cleaved product.
